(19) United States
(12) Patent Application Publication
(10) Pub. No.: US 2023/0263712 A1
(43) Pub. Date: Aug. 24, 2023

(54) AN ORAL CARE ANTIMICROBIAL COMPOSITION, PROCESS FOR PREPARING THE SAME AND METHOD OF USE THEREOF

(71) Applicant: ISP INVESTMENTS LLC, Wilmington, DE (US)

(72) Inventors: Hani M. FARES, Somerset, NJ (US); Yevgeniy TUROVSKIY, Somerville, NJ (US); Solomon Howard JACOBSON, Berkeley Heights, NJ (US); Philip John OTHS, Mendham, NJ (US); Petros GEBRESELASSIE, Whitehouse Station, NJ (US); Joseph TORELLA, Metuchen, NJ (US); Karen WINKOWSKI, Springfield, NJ (US)

(73) Assignee: ISP INVESTMENTS LLC, Wilmington, DE (US)

(21) Appl. No.: 18/012,373

(22) PCT Filed: Jun. 28, 2021
(86) PCT No.: PCT/US21/39392

§ 371 Date: Dec. 22, 2022

Related U.S. Application Data
(60) Provisional application No. 63/046,417, filed on Jun. 30, 2020.
(60) Provisional application No. 63/128,303, filed on Dec. 21, 2020.

(51) Int. Cl. A61K 8/35; A61Q 11/00

(52) U.S. Cl. CPC A61K 8/35; A61Q 11/00; A61K 2800/524; A61K 2800/92; A61K 2800/74

(57) ABSTRACT

An oral care antimicrobial composition comprises about 0.1 to about 99.9 wt. % of at least one compound having the structure of

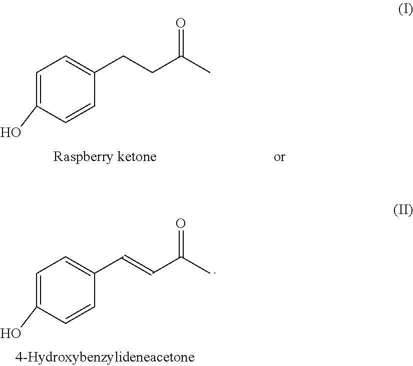

Also disclosed is a process for preparing the composition and methods of use thereof.

### FIELD OF THE INVENTION

The present application relates to an oral care composition, and, more particularly, to an oral care antimicrobial composition comprising (a) Raspberry ketone or 4-Hydroxybenzylideneacetone; and optionally (b) one or more oral care active compounds; and (c) a second antimicrobial compound.

### BACKGROUND OF THE INVENTION

Commercial use products are generally designed to have a substantial shelf life. The products need to be manufactured at one site, transported possibly over a considerable distance to a depot or other storage facility prior to further transport to a point of sale. The product may then spend considerable time on a retailer's shelf prior to purchase and further storage by the user whether for individual use or use in, for example, a workplace, institution or the like. Storage typically takes place under uncontrolled conditions including considerable variation in temperature. In order to keep bacterial and fungal growth in such products at an acceptable level it is conventional practice for the products to contain preservatives and preservative boosters. Even though a variety of conventional preservatives are available for use in oral care products, many of them are not desired by consumers. The concentration of conventional preservatives can be significantly reduced by combining an antimicrobial booster with various actives commonly used in oral care products. Moreover, such a combination can facilitate the antimicrobial activity of the actives within the oral cavity. The appropriate combination of antimicrobial compounds with oral care actives needs to be selected based on its efficacy and its acceptability in the product.

Oral care compositions such as dentifrices, denture adhesives, buccal tapes and chewing gums usually contain one or more oral care active compounds including bactericidal compounds, e.g., triclosan or thymol, and flavorants, e.g., menthol, thymol and essential oils. Chemistries that can boost antimicrobial performance of known actives are highly desired.

There are multiple benefits in using a combination of antimicrobials, especially if they act synergistically with one another. Firstly, the use of such combination greatly reduces the risk of microorganisms developing resistance to the antimicrobials. Additionally, synergistically acting antimicrobials can be used effectively in lower concentrations thus reducing the cost. Finally, a synergistically acting multi-component antimicrobial is likely to be active versus a broader range of microorganisms, or to provide a faster kill rate, compared to each component acting individually. For these reasons, identifying synergistic combinations of antimicrobials is critical for development of effective antimicrobial solutions.

US Publication 20080317681 discloses a composition used as a chewing gum or confectionary for removing stains and microbes from teeth of warm-blooded animals having a stain removing complex containing a stain removing agent, a cyclodextrin compound, and optionally a gum base.

US Publication 20190030107A1 discloses a periodontal adjunct composition containing potassium hydroxide, a metal salt, a phenolic compound, hydrogen receptor antagonist and an essential oil.

US Publication 20160000679A1 discloses a malodor eliminating composition comprising a lactone, a phenolic compound, a malodor masking compound and diol solvent.

PCT Application 2020043269A1 discloses an antimicrobial mixture comprising an antimicrobial agent comprising a substituted acetophenone derivative (I) or its salt, and an antimicrobial agent.

JP publication 2004331505A discloses a dentifrice composition comprising saccharin sodium and raspberry ketone.

JP Patent 03065947B2 discloses a perfume composition for an oral cavity including furaneol and/or raspberry ketone.

JP publication 2004018431A discloses raspberry ketone in an oral fragrance composition.

JP publication 2013170143A discloses raspberry ketone in a perfume composition for an oral cavity.

In view of the foregoing, still there is a need for an improved antimicrobial composition for an aqueous or non-aqueous based end-user oral care composition for reducing, or inhibiting, or preventing microbial growth, comprising adding suitable or effective amounts of antimicrobial compositions into the desired end-user products.

The inventors of the present application determined that oral care actives alone and antimicrobial compounds alone do not individually provide fast antimicrobial kinetic action. However, combinations of oral care actives and antimicrobials at selective concentrations provide unexpected synergistic antimicrobial action.

Accordingly, it is an objective of the present invention to provide an antimicrobial composition comprising (i) raspberry ketone or 4-hydroxybenzylideneacetone; and optionally (ii) at least one or more oral care active compounds; and (iii) one or more second antimicrobial compounds. According to another objective of the present application, there is provided synergistic antimicrobial compositions comprising raspberry ketone or 4-Hydroxybenzylideneacetone, one or more oral care actives and a second antimicrobial compound to kill or inhibit the growth of microorganisms in various aqueous and non-aqueous based end user oral care compositions.

### SUMMARY OF THE INVENTION

The primary aspect of the present application is to provide an oral care antimicrobial composition comprising about 0.1 to about 99.9 wt. % of at least one compound having the structure of

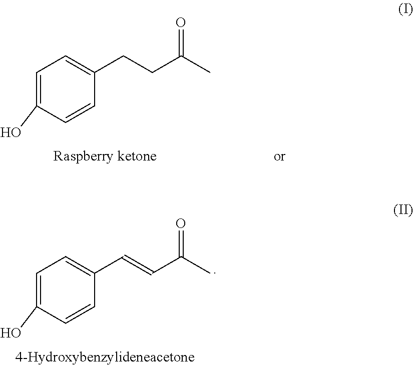

Another non-limiting aspect of the present application discloses an oral care antimicrobial composition comprising: about 0.1 to about 99.9 wt. % of one or more oral care active compounds selected from the group consisting of 4-isopropyl-3-methyl phenol (IPMP), thymol, menthol, chlorhexidine digluconate, cetylpyridinium chloride, sodium fluoride, stannous fluoride, triclosan, methyl salicylate, hydrogen peroxide, potassium nitrate, sodium benzoate, potassium sorbate, benzyl alcohol, methylparaben, benzoic acid, propylparaben, phenoxyethanol, benzalkonium chloride, ethyl paraben, sodium salicylate, benzisothiazolinone, o-phenylphenol, butylated hydroxytoluene (BHT), 2-bromo-2-nitropropane-1,3-diol, sodium hydroxymethylglycinate, diazolidinyl urea, methylisothiazolinone, methylchloroisothiazolinone, sodium hypochlorite, formic acid, sodium ortho-phenylphenate and glucose oxidase.

Another aspect of the present application discloses that the oral care antimicrobial composition further comprises one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts.

Yet another aspect of the present application discloses that the oral care antimicrobial composition can be an aqueous composition or a non-aqueous composition.

Another aspect of the present application provides a process for preparing an oral care antimicrobial composition, wherein the process comprises the steps of mixing: (a) about 0.1 to about 99.9 wt. % of Raspberry ketone or 4-Hydroxybenzylideneacetone; (b) about 0.1 to about 99.9 wt. % of one or more active compounds selected from the group consisting of 4-isopropyl-3-methyl phenol (IPMP), thymol, menthol, chlorhexidine digluconate and cetylpyridinium chloride and combinations thereof; (c) about 0.1 to about 99.9 wt. % of one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts; and (d) about 0.1 to about 99.9 wt. % of at least one or more orally acceptable ingredients.

Another aspect of the present application provides a process for preparing oral care antimicrobial composition, wherein the process comprises the steps of mixing the above-described components (a), (b) and (c) for killing or inhibiting the growth of strains selected from the group consisting of Streptococcus mitis, Streptococcus oxalis, Streptococcus sanguis, Streptococcus gordonii, Staphylococcus aureus, Staphylococcus epidermidis, Streptococcus muris-ratti, Streptococcus cricetus, Streptococcus mutans, Streptococcus sobrinus, Streptococcus sobrinus, Streptococcus macacae, Streptococcus ferus, Neisseria sicca, Veillonella spp, Fusobacterium spp., Actinomyces spp., Corynebacterium spp., Lactobacilli spp., and Prevotella spp.

In another aspect, a method of killing or inhibiting the growth of bacteria and fungi in an aqueous or non-aqueous end-user oral care products that are susceptible to growth of microorganisms comprises incorporating the oral care antimicrobial composition of the present application in an amount ranging from about 0.01 wt. % to 15.0 wt. % into such products.

In yet another aspect, the present application provides a method of preserving an oral care composition comprising adding to the composition an effective amount of (a) about 0.1 to about 99.9 wt. % of Raspberry ketone or 4-Hydroxybenzylideneacetone; (b) about 0.1 to about 99.9 wt. % of one or more oral care active compounds selected from the group consisting of 4-isopropyl-3-methyl phenol (IPMP), thymol, menthol, chlorhexidine digluconate, cetylpyridinium chloride, sodium fluoride, stannous fluoride, triclosan, methyl salicylate, hydrogen peroxide, potassium nitrate, sodium benzoate, potassium sorbate, benzyl alcohol, methylparaben, benzoic acid, propylparaben, phenoxyethanol, benzalkonium chloride, ethyl paraben , sodium salicylate, benzisothiazolinone, o-phenylphenol, butylated hydroxytoluene (BHT), 2-bromo-2-nitropropane-1,3-diol, sodium hydroxymethylglycinate, diazolidinyl urea, methylisothiazolinone, methylchloroisothiazolinone, sodium hypochlorite, formic acid, sodium ortho-phenylphenate and glucose oxidase; (c) about 0.1 to about 99.9 wt. % of one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts; and (d) about 0.1 to about 99.9 wt. % of at least one or more orally acceptable ingredients.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

In a non-limiting embodiment, the present application discloses an oral care antimicrobial composition comprising about 0.1 to about 99.9 wt. % of at least one compound having the structure of

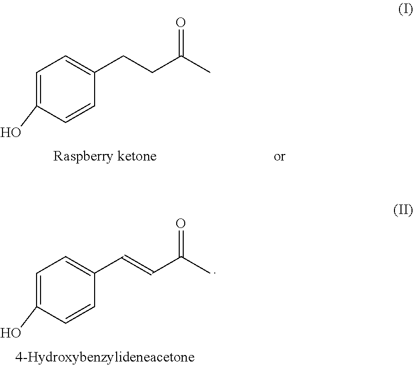

The term “antimicrobial”/“preservative” refers to a substance capable of killing or inhibiting the growth of microorganisms, including but not limited to bacteria and fungi.

As used herein, “Raspberry ketone”, also known as 4-(4-hydroxyphenyl) butan-2-one (HPB), has a CAS No: 5471-51-2. It has been used as an aroma chemical in perfume industries, food industries and in compositions for weight loss with improved taste. Raspberry ketone was described for the first time as a characteristic component of Raspberry flavour (H. Schinz et. al. Helv. Chim. Acta. 1957, 40, 1839).

As used herein, “4-Hydroxybenzylideneacetone”, also known as (E)-4-(4-hydroxyphenyl) but-3-en-2-one has a CAS No. 3160-35-8. It is a precursor for raspberry ketone synthesis.

As used herein, the term “oral care antimicrobial composition” includes, but is not limited to, toothpowders, toothpastes, dentifrices, tooth gel, subgingival gel, mouth-rinse, mousse, foam, denture product, mouth-spray, chewable tablet, dissolvable film, chewing gums, lozenges and denture cleansing formulations suitable for administration or application to the oral cavity a human or animal subject for enhancing the health, hygiene or appearance of the subject.

As used herein, the term “oral care actives” includes bactericidal agents, natural and synthetic agents used in dentistry to inhibit bacterial growth.

In some embodiments, the suitable range of raspberry ketone or 4-hydroxybenzylideneacetone for the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75wt. %; or from about 75 wt. % to about 80 wt. %; or from about 80 wt. % to about 85 wt. %; or from about 85 wt. % to about 90 wt. %; or from about 90 wt. % to about 95 wt. %; or from about 95 wt. % to about 99.9 wt. % based on the total weight of the oral care antimicrobial composition.

As used herein, oral care actives can be selected from the group consisting of 4-isopropyl-3-methyl phenol (IPMP), thymol, menthol, chlorhexidine digluconate, cetylpyridinium chloride, sodium fluoride, stannous fluoride, triclosan, methyl salicylate, hydrogen peroxide, potassium nitrate, sodium benzoate, potassium sorbate, benzyl alcohol, methylparaben, benzoic acid, propylparaben, phenoxyethanol, benzalkonium chloride, ethyl paraben , sodium salicylate, benzisothiazolinone, o-phenylphenol, butylated hydroxytoluene (BHT), 2-bromo-2-nitropropane-1,3-diol, sodium hydroxymethylglycinate, diazolidinyl urea, methylisothiazolinone, methylchloroisothiazolinone, sodium hypochlorite, formic acid, sodium ortho-phenylphenate and glucose oxidase and the like, and mixtures of two or more of these compounds.

In some embodiments, the suitable range of oral care actives for the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. %; or from about 90 wt. % to about 95 wt. %; or from about 95 wt. % to about 99.9 wt. % based on the total weight of the oral care antimicrobial composition.

As used herein, the second antimicrobial compound(s) refers to an antimicrobial compound(s) selected from the group including, but not limited to, alcohols, diols, organic acids or fatty acids, glycerins, caprylates, aldehydes, terpenes, terpenoids, essential oils, peptides, glucosides, enzymes, amino acids and their esters, sclerolide, sclareol and Camelia sinensis extracts and other conventional preservatives that are known to a person skilled in the pertinent art.

According to the present application, non-limiting examples of suitable antimicrobial alcohols useful herein include ethanol and diols. Diols useful herein include, but are not limited to, propanediol, butanediol, pentanediol, hexanediol, octanediol, nonanediol, decanediol and dodecanediol.

In another non-limiting embodiments, it is contemplated to use diols having a carbon chain length of from 3 to 12 atoms, including but not limited to, propane-1,2-diol, propane-1,3-diol, butane-1,2-diol, butane-1,3-diol, butane-1,4-diol, 2-methylpropane-1,2-diol, 2-methylpropane-1,3-diol, pentane-1,2-diol, pentane-1,3-diol, pentane-1,4-diol, pentane-1,5-diol, pentane-2,3-diol, pentane-2,4-diol, 2-methyl-pentane-2,4-diol, hexane-1,2-diol, hexane-1,3-diol, hexane-1,4-diol, hexane-1,5-diol, hexane-1,6-diol, hexane-2,3-diol, hexane-2,4-diol, hexane-2,5-diol, hexane-3,4-diol, heptane-1,2-diol, heptane-1,3-diol, heptane-1,4-diol, heptane-1,5-diol, heptane-1,6-diol, heptane-1,7-diol, heptane-2,3-diol, heptane-2,4-diol, heptane-2,5-diol, heptane-2,6-diol, heptane-3,4-diol, heptane-3,5-diol, octane-1,2-diol, octane-1,3-diol, octane-1,4-diol, octane-1,5-diol, octane-1,6-diol, octane-1,7-diol, octane-1,8-diol, octane-2,3-diol, octane-2,4-diol, octane-2,5-diol, octane-2,6-diol, octane-2,7-diol, octane-3,4-diol, octane-3,5-diol, octane-3,6-diol and octane-4,5-diol, nonane-1,9 diol, decane-1,2-diol, decane-1,10-diol, hexadecan-1-ol, and dodecane-1,12-diol.

In another non-limiting embodiment, the diols having a length of from 3 to 12 carbon atoms, include, but are not limited, to pentane-1,2-diol, hexane-1,2-diol, and octane-1,2-diol.

In another non-limiting embodiment, the organic acids or fatty acids or their salts or esters can be selected from acids having a carbon chain length of from 2 to 25 atoms such as carboxylic acids, and wherein the carboxylic acids or fatty acids are selected from the group including, but not limited to, propionic acid, acetic acid, benzoic acid, malonic acid, succinic acid, fumaric acid, maleic acid, adipic acid, lactic acid, stearic acid, levulinic acid, anisic acid, cinnamic acid, sorbic acid or tartaric acid, malic acid, gluconic acid, citric acid, caproic acid, perillic acid, phytic acid, salicylic acid, undecylenic acid, and the other acids include ascorbic acid, caprylhydroxamic acid, and sorbohydroxamic acid. These acids recited herein further enhance the antibacterial activity while not negatively affecting the quality of the end-user products in terms of their taste, texture, color and odor in which they are applied or engaged.

In another non-limiting embodiment, the organic acids or fatty acids or their salts or esters can be selected from carboxylic acids having from 2 to 25 carbon atoms, including but not limited to, citric acid, stearic acid, benzoic acid, anisic acid, cinnamic acid, phytic acid, sorbic acid, levulinic acid and other acids include caprylhydroxamic acid.

In another non-limiting embodiment, the glycerins useful herein include, but are not limited to, ethylhexylglycerin, butylglycerin, pentylglycerin, hexylglycerin, heptylglycerin, octylglycerin and cyclohexylglycerin.

In still another non-limiting embodiment, the caprylates useful herein include, but are not limited to, glyceryl mono-di caprylate, propylene mono-di caprylate, glyceryl caprylate, sorbitan caprylate, glyceryl undecylenate and glyceryl caprylate/caprate, isosorbide caprylate/caprate and stearyl caprylate.

Yet another non-limiting embodiment discloses to employ aldehydes including, but not limited to, cinnamaldehyde, salicylaldehyde, veratraldehyde, benzaldehyde, butyraldehyde, propionaldehyde, acetaldehyde, and pyruvaldehyde.

The terpenes and terpenoids useful herein for the purposes of the present application include, but are not limited to, citral, pinene, nerol, b-ionone, geraniol, carvacrol, eugenol, carvone, terpeniol, anethole, camphor, menthol, limonene, nerolidol, farnesol, phytol, carotene, squalene, thymol, tocotrienol, perillyl alcohol, bomeol, myrcene, simene, carene, terpenene, tropolone, hinokitiol and linalool.

The essential oils useful herein include, but are not limited to, anise oil, lemon oil, orange oil, oregano, rosemary oil, wintergreen oil, thyme oil, lavender oil, clove oil, hops, tea tree oil, citronella oil, wheat oil, barley oil, lemongrass oil, cedar leaf oil, cedar wood oil, cinnamon oil, fleagrass oil, geranium oil, sandalwood oil, violet oil, cranberry oil, eucalyptus oil, vervain oil, peppermint oil, gum benzoin, basil oil, fennel oil, fir oil, balsam oil, menthol, ocmea origanum oil, Hydrastis canadensis oil, berberidaceae daceae oil, ratanhia oil, curcuma longa oil, sesame oil, macadamia nut oil, evening primrose oil, spanish sage oil, spanish rosemary oil, coriander oil, thyme oil, pimento berries oil, rose oil, bergamot oil, rosewood oil, chamomile oil, sage oil, clary sage oil, cypress oil, sea fennel oil, frankincense oil, ginger oil, grapefruit oil, jasmine oil, juniper oil, lime oil, mandarin oil, marjoram oil, myrrh oil, neroli oil, patchouli oil, pepper oil, black pepper oil, petitgrain oil, pine oil, rose otto oil, spearmint oil, spikenard oil, bitter almond oil, palmarosa oil and vetiver oil.

As used herein, the conventional preservative compounds for the purposes of the present application are selected from the group including, but not limited to, benzoic acid and its sodium salt such as benzoic acid, sodium benzoate; salts of benzoic acid such as ammonium benzoate, butyl benzoate, calcium benzoate, ethyl benzoate, isobutyl benzoate, isopropyl benzoate, magnesium benzoate, MEA-benzoate, methyl benzoate, phenyl benzoate, potassium benzoate, propyl benzoate; propanoic acid and its salts such as propionic acid, ammonium propionate, calcium propionate, magnesium propionate, potassium propionate, sodium propionate; salicylic acid and its salts such as salicylic acid, calcium salicylate, magnesium salicylate, MEA-salicylate, sodium salicylate, potassium salicylate, TEA-salicylate; hexa-2,4-dienoic acid and its salts such as sorbic acid, calcium sorbate, sodium sorbate, potassium sorbate; biphenyl-2-ol as o-phenylphenol; inorganic sulphites and hydrogen sulphites such as sodium sulfite, ammonium bisulfite, ammonium sulfite, potassium sulfite, potassium hydrogen sulfite, sodium bisulfite, sodium meta bisulfite, potassium meta bisulfite; chlorobutanol; 4-hydroxybenzoic acid and its salts and esters other than the esters of isopropyl, isobutyl, phenyl, benzyl and pentyl such as 4-hydroxybenzoic acid, methylparaben, potassium ethylparaben, potassium paraben, sodium methylparaben, sodium ethylparaben, ethylparaben, sodium paraben, potassium methylparaben, calcium paraben; butyl 4-hydroxybenzoate and its salts and propyl 4-hydroxybenzoate and its salts such as butylparaben, propylparaben, sodium propoylparaben, sodium butylparaben, potassium butylparaben, potassium propylparaben; 3-acetyl-6-methylpyran-2,4(3H)- dione and its salts such as dehydroacetic acid, sodium dehydroacetate; formic acid, sodium formate; 3,3′-dibromo-4,4′-hexamethylene dioxydibenzamidine and its salts (including isethionate) such as dibromohexamidine isethionate; thimerosal; phenylmercuric salts such as phenyl mercuric acetate, phenyl mercuric benzoate; undec-10-enoic acid and its salts such as undecylenic acid, potassium undecylenate, sodium undecylenate, calcium undecylenate, MEA-undecylenate, TEA-undecylenate; 5-pyrimidinamine 1,3-bis(2-ethylhexyl)hexahydro-5-methyl- such as hexetidine; 5-bromo-5-nitro-1,3-dioxane; bronopol such as 2-bromo-2-nitropropane-1,3-diol; 2,4-dichlorobenzyl alcohol; 1-(4-chlorophenyl)-3-(3,4-dichlorophenyl)urea such as triclocarban; chlorocresol such as p-chloro-m-cresol, 5-chloro-2-(2,4-dichlorophenoxy)phenol such as triclosan; chloroxylenol; N,N″-methylenebis[N′-[3-(hydro xymethyl)-2,5-dioxoimidazolidin-4-yl]urea] such as imidazolidinyl urea; polyhexamethylene biguanide hydrochloride such as polyaminopropyl biguanide; 2-phenoxyethanol; methenamine;1-(4-chlorophenoxy)-1-(imidazo 1-1-yl)-3,3-dimethylbutan-2-one such as climbazole; 1,3-bis(hydroxymethyl)-5,5-dimethylimidazolidine-2,4-dione such as DMDM hydantoin; benzyl alcohol; 1-hydroxy-4-methyl-6-(2,4,4-tri methylpentyl)-2 pyridon and its monoethanolamine salt such as 1-hydroxy-4-methyl-6-(2,4,4-trimethylpentyl)-2pyridon, piroctone olamine; 2,2′-methylenebis(6-bromo-4-chlorophenol) such as bromochlorophene; 4-isopropyl-m-cresol as o-cymen-5-ol; mixture of 5-chloro-2-methyl-isothiazol-3(2H)-one and 2-methylisothiazol-3(2H)-one with magnesium chloride and magnesium nitrate as methylchloroisothiazolinone and methylisothiazolinone; 2-chloroacetamide; N,N′-bi s(4-chlorophenyl)-3,12-diimino-2,4,11,13-tetraazatetradeca nediamidine and its digluconate, diacetate and dihydrochloride such as chlorhexidine, chlorhexidine diacetate, chlorhexidine digluconate, chlorhexidine dihydrochloride; 1-phenoxypropan-2-ol such as phenoxyisopropanol; alkyl (C12-C22) trimethyl ammonium bromide and chloride such as behentrimonium chloride, cetrimonium bromide, cetrimonium chloride, laurtrimonium bromide, laurtrimonium chloride, steartrimonium bromide, steartrimonium chloride; 4,4-dimethyl-1,3-oxazolidine; N-(hydroxymethyl)-N-(dihydroxymethyl-1,3-dioxo-2,5-imidazolidinyl-4)-N′-(hydroxymethyl)urea such as diazolidinyl urea; benzenecarboximidamide 4,4′-(1,6-hexanediylbis(oxy))bis- and its salts (including isothionate and p-hydroxybenzoate) such as hexamidine, hexamidine diisethionate, hexamidine diparaben, hexamidine paraben; glutaraldehyde (pentane-1,5-dial) such as glutaral; 5-ethyl-3,7-dioxa-1-azabicyclo[3.3.0] octane such as 7-ethylbicyclooxazolidine; 3-(p-chlorophenoxy)-propane-1,2-diol such as chlorphenesin; sodium hydroxymethylamino acetate such as sodium hydroxymethylglycinate; silver chloride deposited on titanium dioxide; benzethonium chloride such as benzenemethanaminium,N,N-dimethyl-N-[2-[2-[4-(1,1,3,3,-tetramethylbutyl)phenoxy]ethoxy]ethyl]-chloride; benzalkonium chloride, benzalkonium bromide, benzalkonium saccharinate; methanol, (phenylmethoxy) such as benzylhemiformal; 3-iodo-2-propynylbutylcarbamate; 2-methyl-2H-isothiazol-3-one; ethyl lauroyl arginate HCl; citric acid (and) silver citrate such as 1,2,3-propanetricarboxylic acid, 2-hydroxy-, monohydrate and 1,2,3-propanetricarboxylic acid, 2-hydroxy-silver(1+) salt, monohydrate; and 4-(3-ethoxy-4-hydroxyphenyl)bu-tan-2-one.

Furthermore, according to the present application, other suitable antimicrobial agents useful herein include peptides, glucosides, enzymes, amino acids and their esters, sclerolide, sclareol, Camellia sinensis and plant extracts that are capable of killing or inhibiting microorganism known to a person skilled in the pertinent art.

In some embodiments, the suitable range of the one or more second antimicrobial compounds of the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75wt. %; or from about 75 wt. % to about 80 wt. %; or from about 80 wt. % to about 85 wt. %; or from about 85 wt. % to about 90 wt. %; or from about 90 wt. % to about 95 wt. %; or from about 95 wt. % to about 99.9 wt. % based on the total weight of the oral care antimicrobial composition.

According to a non-limiting embodiment of the present application, the oral care antimicrobial composition is used for dental care including tooth whitening, enamel repair, activity against gingivitis and plaque or for killing or inhibiting the growth of bacteria. According to a non-limiting embodiment of the present application, the oral care antimicrobial composition is used for killing or inhibiting the growth of strains selected from the group consisting of Streptococcus mitis, Streptococcus oxalis, Streptococcus sanguis, Streptococcus gordonii, Staphylococcus aureus, Staphylococcus epidermidis, Streptococcus muris-ratti, Streptococcus cricetus, Streptococcus mutans, Streptococcus sobrinus, Streptococcus sobrinus, Streptococcus macacae, Streptococcus ferus, Neisseria sicca, Veillonella spp, Fusobacterium spp., Actinomyces spp., Corynebacterium spp., Lactobacilli spp., and Prevotella spp.

According to a non-limiting embodiment of the present application, the oral care antimicrobial composition further comprises an orally acceptable ingredient selected from the group consisting of an anti-inflammatory agent, a sialagogue agent, an anti-caries agent, an anti-gingivitis agent, a pain reliving agent, an antioxidant, an enzyme, a flavor, a cooling agent, a sweetener and mixtures thereof.

According to another non-limiting embodiment of the present application, the oral care antimicrobial composition can provide a synergistic effect of the antimicrobial composition in various aqueous and non-aqueous oral care end-user applications, and wherein the synergistic index (SI) value greater than 0.1 to less than 1.

Another non-limiting embodiment of the present application discloses that the oral care antimicrobial composition can advantageously be used in other personal care compositions, and wherein, the compositions can be an aqueous or non-aqueous based end-user composition. Aqueous and non-aqueous based end-user applications include, but are not limited to, personal care or cosmetic products, toiletry products, skin care products, hair care products, household & cleaning products, soap and bath products, industrial and institutional cleaning products, disinfecting products, wound care products, sanitary products, agricultural compositions, textile products, coating products and laundry products.

In some non-limiting embodiments, the present application discloses that a suitable range of the antimicrobial composition for killing or inhibiting the growth of bacteria and fungi in the aqueous or non-aqueous based end-user products can be varied from about 0.01 wt. % to about 0.1 wt. %, or from about 0.1 wt. % to about 1 wt. %, or from about 1 wt. % to about 2.5 wt. %, or from about 2.5 wt. % to about 5 wt. %, or from about 5 wt. % to about 10 wt. %; or from about 10 wt. % to about 15 wt. % based on the total weight of the aqueous or non-aqueous personal care composition.

In a non-limiting embodiment, the present application discloses an antimicrobial composition comprising (a) about 0.1 to about 99.9 wt. % of Raspberry ketone or 4-Hydroxybenzylideneacetone; (b) about 0.1 to about 99.9 wt. % of one or more oral care actives selected from the group consisting of 4-isopropyl-3-methyl phenol (IPMP), thymol, menthol, chlorhexidine digluconate and cetylpyridinium chloride and combinations thereof; and (c) about 0.1 to about 99.9 wt. % of at least one or more orally acceptable ingredients.

Another embodiment of the present application provides a process for preparing an oral care antimicrobial composition, wherein the process comprises the steps of mixing: (a) about 0.1 to about 99.9 wt. % of Raspberry ketone or 4-Hydroxybenzylideneacetone; (b) about 0.1 to about 99.9 wt. % of one or more active compounds selected from the group consisting of 4-isopropyl-3-methyl phenol (IPMP), thymol, menthol, chlorhexidine digluconate and cetylpyridinium chloride and combinations thereof; and (c) about 0.1 to about 99.9 wt. % of one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts; and (d) about 0.1 to about 99.9 wt. % of at least one or more orally acceptable ingredients.

Yet another embodiment of the present application provides a method of preserving an oral care composition comprising adding an effective amount of (a) about 0.1 to about 99.9 wt. % of Raspberry ketone or 4-Hydroxybenzylideneacetone; (b) about 0.1 to about 99.9 wt. % of one or more oral care active compounds selected from the group consisting of 4-isopropyl-3-methyl phenol (IPMP), thymol, menthol, chlorhexidine digluconate, cetylpyridinium chloride, sodium fluoride, stannous fluoride, triclosan, methyl salicylate, hydrogen peroxide, potassium nitrate, sodium benzoate, potassium sorbate, benzyl alcohol, methylparaben, benzoic acid, propylparaben, phenoxyethanol, benzalkonium chloride, ethyl paraben, sodium salicylate, benzisothiazolinone, o-phenylphenol, butylated hydroxytoluene (BHT), 2-bromo-2-nitropropane-1,3-diol, sodium hydroxymethylglycinate, diazolidinyl urea, methylisothiazolinone, methylchloroisothiazolinone, sodium hypochlorite, formic acid, sodium ortho-phenylphenate and glucose oxidase; (c) about 0.1 to about 99.9 wt. % of one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts; and (d) about 0.1 to about 99.9 wt. % of at least one or more orally acceptable ingredients.

Further, certain aspects of the present application are illustrated in detail by way of the following examples. The examples are given herein for illustration of the application and are not intended to be limiting thereof.

### EXAMPLES

### Example 1: Synergy Between 4-(4-hydroxyphenyl)butan-2-one (HPB) and Oral Care Active Compounds Using the Checkerboard Assay

A combination of raspberry ketone with either IPMP or thymol exhibited strong synergistic activity (SI ≤0.5016 and ≤0.625 respectively). Menthol, chlorhexidine digluconate and cetylpyridinium chloride (CPC) also acted synergistically with raspberry ketone (SI ≤0.75; Synergy with eucalyptol was not detected using a standard synergy assay described below, as eucalyptol did not exhibit any efficacy under these conditions even when it was solubilized with 2% of Pluronic F-127 (MIC >2%, Table 1).

Synergy between of 4-(4-hydroxyphenyl)butan-2-one (HPB) and various known antimicrobial compounds commonly used in oral care was demonstrated versus Streptococcus mutans UA159. The synergistic antimicrobial effect was determined using the commonly accepted method described by Kull A. C, Eisman, P. C. Sylwestrowicz, H. D. and Mayer, R. L. 1961. Applied Microbiology, 9:538-541

Brief description of the assay: Standard checkerboard approach was used to make all the dilutions. HPB (compound A) solution was prepared in DMSO and then serially diluted using a 96-well dilution plate. Automated liquid handler was then used to stamp multiple assay plates out of this dilution plate. Each well of the assay plate was then supplemented with a predetermined concentration of the second antimicrobial (compound B). The final concentration of DMSO was kept constant across the plate for each assay, not exceeding 3% (w/w). Then, S. mutans cell suspension in Todd Hewitt Broth supplemented with yeast extract (THYEB) was added to the appropriate wells of the assay plate with the final cell density being 106 cfu/ml. The assay plates were incubated for 48 hours at 37° C. in the atmosphere containing 5% CO₂ prior to being evaluated.

The lowest concentration of each compound (or a mixture of the two compounds) to inhibit visible growth was taken as the minimum inhibitory concentration (MIC). The MIC was taken as the end point of activity. End points for the combination of compound A (HPB) and compound B were then compared to the end points for each compound being used individually.

The following formula was then utilized to calculate the Synergy Index (SI) between compounds A and B:

Synergy Index(SI)=Qa/QA+Qb/QB

### Wherein:

QA is the Minimal Inhibitory Concentration (MIC) of compound A in ppm, acting alone, which produced an end point.
Qa is the MIC of compound A in ppm, when compound A is being used in combination with compound B, which produced an end point.
QB is the MIC of compound B in ppm, acting alone, which produced an end point.
Qb is the MIC of compound B in ppm, when the compound B is being used in combination with compound A, which produced an end point

A synergistic effect is indicated if the Synergy Index is below one (SI <1). An additive effect is indicated if the Synergy Index is equal to one (SI =1). An antagonistic effect is indicated if synergy index is greater than one (SI >1).

The results presented in the Table 1 comprise the following parameters:
(i) The end-point activity for compound A (HPB) alone in ppm measured as MIC (QA)
(ii) for compound A (HPB) in the combination with the compound B (Qa), (iii) for compound B alone (QB), (iv) for compound B in the combination with compound A (Qb), (v) the synergy index (SI) based on the formula SI=Qa/QA+Qb/QB

TABLE 1
Synergy data vs. S. mutans, generated using the checkerboard microplate assay
with the compound A being 4-(4-hydroxyphenyl) butan-2-one (HPB)

| | | MIC in ppm | | | | |
|---|---|---|---|---|---|---|
| | | | Compound B | | Compound B | |
| | Compound B | HPB Qa | Qb | HPB QA | QB | SI index |
| S. | Isopropyl | 125 | 250 | ≥8,000 | 500 | ≤0.5016 |
| mutans | Methylphenol | | | | | |
| | (IPMP) | | | | | |
| | Thymol | 4,000 | 15.6 | ≥8,000 | 125 | ≤0.625 |
| | Menthol | 4,000 | 1,000 | ≥8,000 | ≥4,000 | ≤0.75 |
| | Chlorhexidine | 4,000 | 0.4 | ≥8,000 | 1.6 | ≤0.75 |
| | digluconate | | | | | |
| | Cetylpyridinium | 2,000 | 0.3 | ≥8,000 | 0.6 | ≤0.75 |
| | chloride | | | | | |
| | monohydrate | | | | | |

### Example 2

In a suitable vessel, fitted with a propeller type mixing blade, dissolve the raspberry ketone in propylene glycol and glycerin under moderate agitation. Then mix in the sorbitol 70% solution and water and dissolve the poloxamer under moderate to high agitation. The sodium fluoride and sodium saccharin were then dissolved under moderate agitation. The rest of the ingredients were added, using moderate agitation, in the following order: dibasic sodium phosphate dihydrate, monobasic sodium phosphate monohydrate, cetylpyridinium chloride and, flavor oils (peppermint oil and eugenol) in the amounts set forth in Table 2.

TABLE 2
Mouthwash Composition

| | Formula 1 | Formula 2 |
|---|---|---|
| | % (w/w) | % (w/w) |
| Water | 64.68 | 64.63 |
| Glycerin | 8.50 | 8.50 |
| Sorbitol, 70% | 15.00 | 15.00 |
| Propylene glycol | 8.00 | 8.00 |
| Poloxamer 407 | 3.00 | 3.00 |
| Raspberry Ketone | 0.500 | 0.500 |
| Peppermint Oil | 0.100 | 0.10 |
| Eugenol | — | 0.050 |
| Sodium Fluoride | 0.050 | 0.050 |
| Sodium Saccharin | 0.030 | 0.030 |
| Dibasic sodium phosphate dihydrate | 0.063 | 0.063 |
| Monobasic sodium phosphate monohydrate | 0.038 | 0.038 |
| Cetylpyridinium chloride monohydrate | 0.040 | 0.040 |
| Totals: | 100.0 | 100.0 |
| pH (as-is): | 7.0 | 7.0 |

What is claimed is:

1. An oral care antimicrobial composition comprising about 0.1 to about 99.9 wt. % of at least one compound having the structure

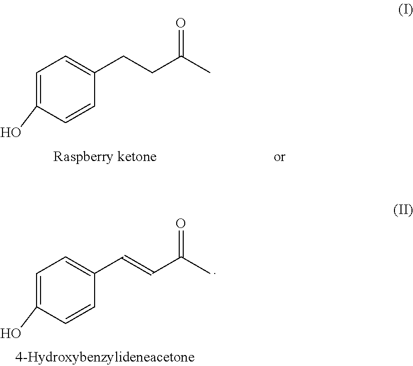

2. The oral care antimicrobial composition according to claim 1, wherein the compound is raspberry ketone.

3. The oral care antimicrobial composition according to claim 1, further comprising about 0.1 to about 99.9 wt. % of one or more oral care active compounds selected from the group consisting of 4-isopropyl-3-methyl phenol (IPMP), thymol, menthol, chlorhexidine digluconate, cetylpyridinium chloride, sodium fluoride, stannous fluoride, triclosan, methyl salicylate, hydrogen peroxide, potassium nitrate, sodium benzoate, potassium sorbate, benzyl alcohol, methylparaben, benzoic acid, propylparaben, phenoxyethanol, benzalkonium chloride, ethyl paraben, sodium salicylate, benzisothiazolinone, o-phenylphenol, butylated hydroxytoluene (BHT), 2-bromo-2-nitropropane-1,3-diol, sodium hydroxymethylglycinate, diazolidinyl urea, methylisothiazolinone, methylchloroisothiazolinone, sodium hypochlorite, formic acid, sodium ortho-phenylphenate and glucose oxidase.

4. The oral care antimicrobial composition according to claim 1, further comprising about 0.1 to about 99.9 wt. % of one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts.

5. The oral care antimicrobial composition according to claim 1, wherein the composition is used for killing or inhibiting the growth of strains selected from the group consisting of Streptococcus mitis, Streptococcus oxalis, Streptococcus sanguis, Streptococcus gordonii, Staphylococcus aureus, Staphylococcus epidermidis, Streptococcus muris-ratti, Streptococcus cricetus, Streptococcus mutans, Streptococcus sobrinus, Streptococcus sobrinus, Streptococcus macacae, Streptococcus ferus, Neisseria sicca, Veillonella spp, Fusobacterium spp., Actinomyces spp., Corynebacterium spp., Lactobacilli spp., and Prevotella spp.

6. The oral care antimicrobial composition according to claim 1, wherein the antimicrobial composition further comprises an orally acceptable ingredient selected from the group consisting of an anti-inflammatory agent, a pain reliving agent, an antioxidant, an enzyme, a flavor, a cooling agent, a sweetener and mixtures thereof.

7. An oral care antimicrobial composition according to claim 1, wherein the composition is used for dental care including tooth whitening, enamel repair, activity against gingivitis and plaque or for killing of or inhibiting the growth of bacteria.

8. The oral care antimicrobial composition according to claim 1, wherein the composition is an aqueous composition or non-aqueous composition.

9. The antimicrobial composition according to claim 1, wherein the composition is formulated as a toothpaste, dentifrice, tooth gel, subgingival gel, tooth powder, mouth-rinse, mousse, foam, denture product, mouth-spray, chewable tablet, dissolvable film or chewing gum.

10. The oral care antimicrobial composition according to claim 1, wherein the composition provides a synergistic effect having a synergistic index (SI) value greater than 0.1 to less than 1.

11. The use of the oral care antimicrobial composition according to claim 7, wherein the amount of said composition employed in an aqueous or non-aqueous based end-user oral care composition is in the range of from about 0.01 wt. % to about 15.0 wt. % of the total composition.

12. A process for preparing an oral care antimicrobial composition, wherein the process comprises the steps of mixing:
(a) about 0.1 to about 99.9 wt. % of Raspberry ketone or 4-Hydroxybenzylideneacetone;
(b) about 0.1 to about 99.9 wt. % of one or more oral care active compounds; and
(c) about 0.1 to about 99.9 wt. % of at least one or more orally acceptable ingredients.

13. A method of killing or inhibiting the growth of bacteria and fungi in an aqueous or non-aqueous based oral care product susceptible to growth of microorganisms comprising incorporating about 0.01 wt. % to 15.0 wt. % of the antimicrobial composition of claim 1 into said product.

14. The process for preparing the oral care antimicrobial composition according to claim 12, further comprising mixing about 0.1 to about 99.9 wt. % of a second antimicrobial compound selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts.

15. An oral care antimicrobial composition comprising:
(a) about 0.1 to about 99.9 wt. % of Raspberry ketone or 4-Hydroxybenzylideneacetone;
(b) about 0.1 to about 99.9 wt. % of one or more active compounds selected from the group consisting of 4-isopropyl-3-methyl phenol (IPMP), thymol, menthol, chlorhexidine digluconate, cetylpyridinium chloride and combinations thereof;
(c) about 0.1 to about 99.9 wt. % of one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts; and
(d) about 0.1 to about 99.9 wt. % of at least one or more orally acceptable ingredients.

16. A method of preserving an oral care composition comprising adding an effective amount of
(a) about 0.1 to about 99.9 wt. % of Raspberry ketone or 4-Hydroxybenzylideneacetone;
(b) about 0.1 to about 99.9 wt. % of one or more oral care active compounds selected from the group consisting of 4-isopropyl-3-methyl phenol (IPMP), thymol, menthol, chlorhexidine digluconate, cetylpyridinium chloride, sodium fluoride, stannous fluoride, triclosan, methyl salicylate, hydrogen peroxide, potassium nitrate, sodium benzoate, potassium sorbate, benzyl alcohol, methylparaben, benzoic acid, propylparaben, phenoxyethanol, benzalkonium chloride, ethyl paraben, sodium salicylate, benzisothiazolinone, o-phenylphenol, butylated hydroxytoluene (BHT), 2-bromo-2-nitropropane-1,3-diol, sodium hydroxymethylglycinate, diazolidinyl urea, methyli sothiazolinone, methylchloroisothiazolinone, sodium hypochlorite, formic acid, sodium ortho-phenylphenate and glucose oxidase;
(c) about 0.1 to about 99.9 wt. % of one or more second antimicrobial compounds selected from the group consisting of alcohols, diols, organic acids including carboxylic acids, glycols, glycerins, caprylates, aldehydes, terpenes, terpenoids, essential oils, peptides, glucosides, enzymes, amino acids and their esters, sclerolide, sclareol, and Camelia sinensis extracts; and
(d) about 0.1 to about 99.9 wt. % of at least one or more orally acceptable ingredients.
